(19) United States
(12) Patent Application Publication
(10) Pub. No.: US 2004/0038946 A1
(43) Pub. Date: Feb. 26, 2004

(54) Fullerene-based drugs targeted to bone

(76) Inventors: Lon J. Wilson, Houston, TX (US); Uri Sagman, Toronto, Ontario (CA); Kenneth Pritzker, Toronto,Ontario (CA)

Correspondence Address: CONLEY ROSE, P.C., P. O. BOX 3267, HOUSTON, TX, 77253-3267, US

(21) Appl. No.: 10/258,774

(22) PCT Filed: Apr. 27, 2001
(86) PCT No.: PCT/US01/13445

(51) Int. Cl. A61K 31/66; A61K 33/44

(52) U.S. Cl. 514/102; 424/125

(57) ABSTRACT

A method for providing bone therapy in a patient in need of bone therapy comprises administering to the patient a pharmaceutically effective amount of a compound comprising a biologically inert carrier, a bone vector; and a therapeutic agent. The bone vector preferably comprises a bisphosphonate, the carrier preferably comprises a fullerene, and more preferably C₆₀, the therapeutic agent preferably comprises fluoride.

### CROSS-REFERENCE TO RELATED APPLICATIONS

The present application claims the benefit of U.S. Provisional Application Serial No. 60/199,970 filed Apr. 27, 2000, which is incorporated herein by reference.

### FIELD OF THE INVENTION

The present invention relates generally to the delivery of therapeutic compounds to bone and more particularly to the use of fallerene molecules to mitigate the toxicity of bone-therapeutic agents. Still more particularly, the present invention relates to the use of fullerenes in conjunction with bone-vectoring compounds and bone-therapeutic agents to yield a bone-targeted, non-toxic bone-therapeutic agent.

### BACKGROUND OF THE INVENTION

Bone is the hard form of connective tissue that makes up most of the skeleton; it consists of an organic component, the cells and matrix, and an inorganic, or mineral component. The matrix contains a framework of collagenous fibers, mainly infused with the mineral component, calcium, which makes bone rigid and strong. Tiny fluid-containing channels, called canaliculi, cross the mineralized bone, facilitating the transfer of calcium from the bone interior to exterior.

There are a total of 206 bones in the human skeleton. Bone serves as a structural frame to support the body; it enables movement by providing a point of attachment for the muscles and by serving as a system of levers; it protects the brain, spinal cord, and the soft internal organs; it houses the blood-forming system (red bone marrow); and it acts as a reservoir for the mineral calcium, which is vital to many body processes.

Living bone is continuously recycled by the processes of bone formation and resorption. During an animal's growth years, bone formation exceeds resorption and the skeletal mass increases. In humans, bone mass reaches a peak between ages 20 and 30 years. After that time, the rate of formation and resorption stabilize the bone mass until age 35 to 40 years, at which time resorption begins to exceed formation, and the total mass slowly decreases. The process of bone turnover in adults is known as remodeling. Up to 15 percent of the total bone mass turns over every year in the remodeling process.

Two major cell types make up bone and are responsible for the remodeling process (ossification): osteoclasts and osteoblasts. Osteoclasts absorb and remove mineralized bone, releasing calcium and phosphate. Osteoblasts assimilate calcium and phosphate to slowly produce crystals, or mineralized bone. As the mineralized bone accumulates and surrounds the osteoblast, that cell slows its activity and becomes an interior osteocyte.

Between the areas of osteoclastic and osteoblastic activity is a cement line containing bone matrix material which delineates the zones of resorption and new bone formation. Bone formation takes place in areas where bone undergoes the greatest stress. Therefore, a bone that is underutilized, such as a leg that is immobilized, is prone to resorption.

Bone remodeling not only alters the architecture of the bone, it also enables the body to regulate the levels of calcium ions in the blood and interstitial fluid. These calcium levels must remain within a fairly narrow range in order to ensure the proper functioning of nerve transmission, the integrity and permeability of cellular membranes, and the ability of the blood to clot. Bone contains about 99 percent of the body's calcium. When fluid calcium levels fall too low, parathyroid hormone stimulates osteoclast activity (causing increased bone resorption) and the subsequent release of calcium into the bloodstream. When fluid calcium levels rise excessively, the hormone calcitonin inhibits resorption (acting against the parathyroid hormone), thereby restricting the release of calcium from the bones. It is necessary to have a healthy intake of calcium to maintain the body's calcium reserve; otherwise, the calcium levels in the body become dependent on the resorption of bone tissue. Vitamin D is also essential, as it makes possible the bodys use of ingested calcium. Estrogen also inhibits bone resorption.

The susceptibility of bone to disease alters with age. Children tend to suffer from abnormal bone development. Young adults are prone to rheumatoid arthritis and spinal difficulties, such as scoliosis. The elderly are vulnerable to metabolic disorders that affect the composition of bone, as well as to osteoarthritis and other joint disorders and to circulatory problems that affect bone health.

Many bone diseases are related to the composition and scale of bone tissue. For example, when a bone has much more bone tissue than average, it is termed osteosclerosis; when there is less, it is called osteopenia. If bone suffers from a lack of mineral content, it is called rickets in children and osteomalacia in adults. The afflicted bones become malleable and vulnerable to deformities. In children, this condition is often the result of vitamin D deficiency. Of all bone diseases, osteoporosis, a generalized osteopenia, is the most common. This disease primarily affects the aged and is more serious in women than men. Osteoporosis is responsible for many of the fractures encountered by the elderly. Another disease that often afflicts the elderly is Paget's disease, characterized by bone deformity and calcium imbalance.

Likewise, bone cells can be killed by a lack of blood supply, this tissue death is termed necrosis or osteonecrosis. It can be brought on by injury, the blockage of an artery, circulatory problems, the administration of corticosteroid hormones for the treatment of another affliction, or by a disease of the metabolic system. Osteomyelitis refers to a bone infection, which can be acquired through an open wound, or from an infection elsewhere in the body. Tumors can also develop in bone tissue. Congenital bone diseases refer to abnormalities which are present at birth; some are genetically transferred but most occur due to problems during pregnancy or delivery. Lastly, bone fractures are the result of a force greater than the strength and resistance of the bone. Age and disease are factors that determine whether a given force will cause a fracture.

The conditions and factors listed above can cause undesired bone loss or a need to replace lost bone through enhanced bone growth. Hence, compositions that mitigate bone loss and/or encourage bone growth are the subject of ongoing research.

For example, various chemical/hormonal treatments have been tried as methods for treating bone disease. Hormones such as estrogen have shown to promote bone growth. Unfortunately, because estrogens have been linked to cancer and other undesirable side effects, they are not widely prescribed for bone therapy.

One recently approved treatment for bone disease is a class of chemicals known as bisphosphonates. Bisphosphonates bind to bone, slowing osteoclasts and allowing new bone to be formed. However, because this effect is temporary, bone mass is not substantially increased in the long term. Because new bone is not formed, bones are left weakened and prone to later injury Hence, bisphosphonates alone are not entirely satisfactory. For all of the foregoing reasons, there remains a need for a suitable compound that inhibits bone resorption and promotes new bone formation so as to produce a net bone gain without adversely affecting the patient.

### SUMMARY OF THE INVENTION

The present invention provides a non-toxic, biologically active composition that is capable of encouraging bone growth while simultaneously inhibiting bone resorption, so as to produce a net bone gain. A preferred embodiment is a bimodal fullerene-based compound that includes a bone-targeting ligand and a bone growth enhancing ligand. In a particularly preferred embodiment, the composition comprises fullerene molecules, sometimes referred to as “buckyballs,” conjugated with both an osteogenic or osteoinductive agent and a bisphosphonate.

### BRIEF DESCRIPTION OF THE FIGURES

A better understanding of the present invention can be obtained when the following detailed description of the preferred embodiment is considered in conjunction with the following drawings, wherein:

FIG. 1 illustrates a typical bisphosphonate;

FIG. 2 is an example of a water-solubilized fullerene suitable for use in the present invention;

FIG. 3 is an example of a water-solubilized bone-targeted fullerene suitable for use in the present invention;

FIG. 4 is an example of a water-solubilized bone-targeted fullerene including a therapeutic agent in accordance with one embodiment of the present invention;

FIG. 5 is a plot of titrant volume added as a function of time during constant composition hap crystal growth inhibition experiments with C₆₀(OH)₃₀;

FIG. 6 is a plot of titrant volume added as a function of time during constant composition hap crystal growth inhibition experiments with C₆₀(OH)₁₆, AMBP;

FIG. 7 is a plot of the rate of HAP crystal growth in the presence of C₆₀(OH)₃₀ and C₆₀(OH)₁₆AMBP as a function of additive concentration; and

FIG. 8 is a plot of Rₒ/R versus concentration for C₆₀(OH)₃₀ and C₆₀(OH)₁₆AMBP.

### DETAILED DESCRIPTION OF THE PREFERRED EMBODIMENTS

The present invention employs fuillerene- or metallofllerene-based materials as diagnostic and therapeutic drugs because they are relatively non-toxic, non-metabolizable, and capable of in vivo delivery diagnostic and/or therapeutic agents. Additionally, with the aid of targeting agents, the present fullerene- and metallofilllerene-based materials are capable of being selectively delivered to specific tissue upon demand. A preferred embodiment is a bimodal fullerene-based compound that includes a bone-targeting ligand and a bone growth enhancing ligand.

The present invention comprises three main components: (1) a vector, (2) a carrier and (3) a therapeutic/diagnostic agent. Preferred embodiments of each of these components are discussed in detail below.

Vector

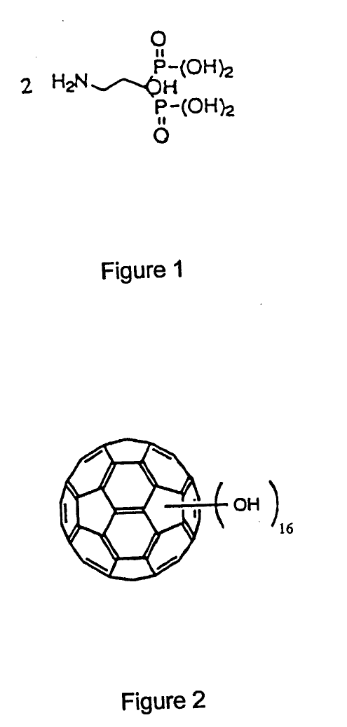

A vector is defined herein as something that targets a specific tissue, in this case bone. In preferred embodiments, bisphosphonates are used as bone-targeting vectors. Bisphosphonates are compounds characterized by two C-P bonds. A representative bisphosphonate is shown in FIG. 1. All bisphosphonates act in a similar manner on bone: they bind permanently to mineralized bone surfaces and inhibit subsequent osteoclastic activity, namely the removal of bone during the process of bone remodeling. Because they reduce the amount of bone tissue degraded during the remodeling cycle, they are sometimes referred to as “antiresorptive agents.” The application of bisphosphonates usually reduces bone loss and, correspondingly, the risk of broken bones, and sometime increases bone mass.

As a group, the bisphosphonates offer several advantages over estrogens in treating osteoporosis. They are bone-tissue specific, have minimal side effects (e.g. nausea, abdominal pain and loose bowel movements), cause no known risk of carcinogenesis, and have antiresorptive efficacy that is equivalent to or greater than estrogens. There is some evidence that the use of bisphosphonates can cause a reduction in incident vertebral fractures.

Carrier

A carrier is defined herein as something that transports other things, in this case a vector and at least one therapeutic/diagnostic agent. Fullerene molecules, sometimes referred to as “buckyballs,” are preferred carriers because they are relatively non-toxic, suitably sized for in vivo applications, biologically inert, and can be made water soluble. Additionally, because of the large number of pi-bonds in fullerenes, they can easily be attached to other molecules, making them an ideal vehicle for trasporting other substances. Still further, it is believed that the spherical shape of certain fullerenes provides a versatile scaffold to which ligands can be attached in a variety of substantially controllable and selectable configurations.

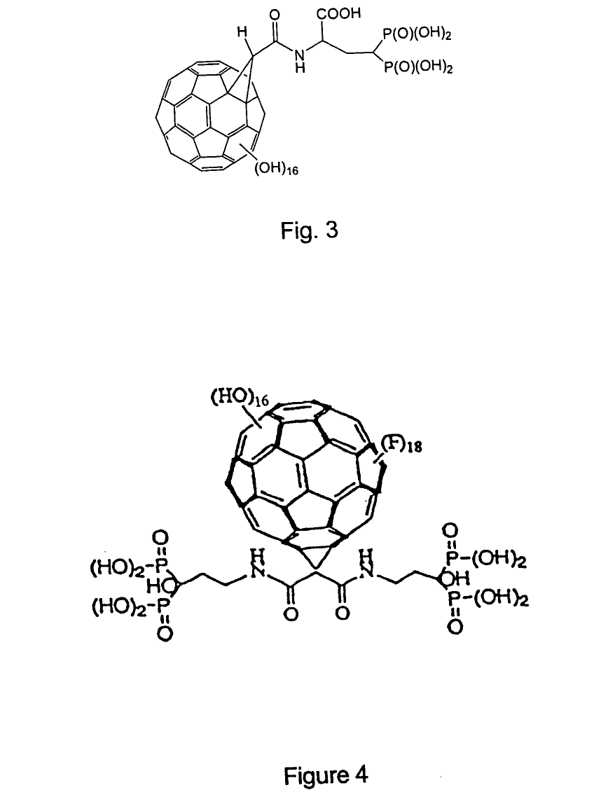

C₆₀, illustrated in FIG. 2, is an example of a fullerene suitable for use in the present invention. Other suitable fullerenes include but are not limited to: C₇₀, C₈₀, and their derivatives, endofullerenes, nanotubes, and higher carbon-number fullerenes. While fullerene molecules are acceptably non-toxic, their use in vivo is limited by their hydrophobicity. Fullerenes are known to be rendered less hydrophobic by the addition of hydroxyl or carboxylic acid groups. In general, 12-16 hydroxyl groups or six carboxyl groups are needed to adequately solubilize C₆₀ and related fullerene molecules. FIG. 3 illustrates an exemplary bisphosphonated hydroxylated fullerene capable of functioning as a bone-targeted carrier.

In a preferred embodiment, vectors and therapeutic/diagnostic agents are externally bound the carrier. In some embodiments, however, it is contemplated that fullerene cages may contain such substances, i.e. therapeutic and/or diagnostic agents. For instance, a radionuclide-containing fullerene derivative could be successfully vectored to diseased sites in bone so as to provide tissue-specific radiation therapy.

Therapeutic and/or Diagnostic Agent

A therapeutic agent is defined herein as a compound that is used to treat disease or alter a medical condition. Because the number of diseases and injuries in bone is large, many therapeutic agents are contemplated for use in the present invention. Additionally, because the therapeutic agents are attached to a carrier, substances that may be too toxic to be trnsported in the body in free form can be used in the present invention.

For example, fluoride anion (F⁻) is known to be an active therapeutic agent for treatment of osteoporosis. While F⁻is too toxic to be administered in free form (e.g. injecting NaF in aqueous solution intravenously), F⁻may be incorporated into a delivery system that releases the agent at the bony site and avoids early, potentially harmful, release in the body before reaching the bony site. Other suitable therapeutic agents may include, but are not limited to: antioxidants, photodynamic therapeutic agents, and biologically derived agents, such as collagen-derived proteins and bone growth factor (bgf).

Alternatively, the bone-vectored fullerenes of the present invention can be used in conjunction with diagnostic agents. Suitable diagnostic agents that can be bound to fullerenes include but are not limited to: magnetic resonance imaging (MRI) contrast agents such as paramagnetic lanthanide or transition metal ion complexes, radiotracers/radioactive metal ions, and the like.

Bone tissue is an especially appealing target for vectored pharmaceuticals because its primary inorganic component, hydroxyapatite (HAP), offers a multitude of binding sites for structurally suitable compounds. Compounds with functional groups such as hydroxyls and carboxylic and phosphonic acids are capable of forming ionic and hydrogen bonds to the mineral portion of bone. The interactions between a bone-vectored compound and the mineralized tissue may be modeled in vitro using HAP crystal growth inhibition studies, whereby compounds with high affinity for HAP bind to the surface of the crystals at kinks and dislocations, blocking crystallization. Using carefully designed experiments, the extent of crystal growth inhibition by a bone-vectored compound can then be used to estimate the compound's affinity for bone tissue in vivo.

Crystal growth inhibition technology is especially important because bone-vectored compounds typically target areas of bone that are undergoing formation and resorption processes (remodeling). The vectored compounds are attracted to the active growth sites of HAP, and thus bind in greatest concentration to the metabolically-active portions of bone. Where bone tissue is diseased, a high rate of bone metabolism exists, and this activity attracts suitably-derivatized compounds to the diseased site.

FIG. 4 illustrates one preferred embodiment of the present invention. An alendronate (a bisphosphonate) is the vector or targeting agent, C₆₀ is the carrier, and fluoride is the therapeutic agent. It will be understood that modifications can be made to composition of the ligands and the carrier, and the relative amounts of each can be varied, so long as each ligand and the carrier can be combined to give an effective, bone-targeted, bone-therapeutic compound. Because the compounds of the present invention are highly tissue-specific, the dose level required for treatment is relatively low. This, in turn, reduces treatment costs and potential harm to non-diseased tissues. Heretofore, no tissue-vectored fullerene derivatives have been reported. The following Example is intended to be illustrative only and are not intended to limit the scope of the present invention.

### EXAMPLE

A bisphosphonate-C₆₀ derivative (C₆₀(OH)₁₆AMBP) is targeted to mineralized bone in vitro. This example does not contain a therapeutic agent, because it is meant to illustrate that fullerene-based materials are capable of being selectively delivered to specific tissue. One or more known therapeutic(s) to a fillerene-based material for bone stimulation may be added by methods known in the art.

Materials

Reagent grade solvents and electrolytes (Fisher) were used without purfication unless stated otherwise. Anhydrous solvents were obtained by distillation from appropriate drying agents under inert atmosphere. Petroleum ether and bromobenzene were each pre-dried with NaSO4 and then refluxed over and distilled from sodium. Benzene, toluene, and ethyl ether were each pre-dried with CaCl2 and then refluxed over and distilled from sodium in the presence of sodium benzophenone ketyl. Chloroform, stabilized with 0.75% ethanol, was used as received. Triethylamine was distilled from KOH under inert atmosphere.

Silica gel (Aldrich, grade 62, 60-200 mesh, 150 Å) was activated at 130° C. for a minimum of 12 hours before use. Sephadex G-25 (Aldrich, 20-80 μL) was equilibrated in DI H2O for 24 h at room temperature prior to use.

Physical and Spectroscopic Methods

Unless otherwise noted, residual solvent signals were used for spectral reference in the 13C and 1H NMR spectra (DMSO-d6, 2.50 and 39.1 ppm; CDCl3, 7.26 and 77.0 ppm; D20, 4.70 ppm). Phosphoric acid (85%, 0 ppm) was used as an external reference for 31P NMR spectra. Signals that were shifted upfield from H3PO4 were assigned positive values; signals downfield from H3PO4 were assigned negative values. For each set of phosphorus NMR data, the upfield or downfield shift is stated for clarity.

Mass spectra were measured on a Finnigan MAT 95 GC-MS analyzer using electron ionization (EI, 70 eV) or atmospheric pressure chemical ionization (APCI). High resolution APCI peak matching spectra were collected using Gramocidin S as the peak reference at 1141.71376 amu in 50/50 CHCl3/MeOH.

Elemental analyses were obtained commercially from Galbraith Laboratories, Inc., Knoxville, Tenn.

Materials and Methods for HAP Crystal Growth Inhibition Studies

Hydroxyapatite seed crystals were prepared from calcium nitrate and potassium dihydrogen phosphate, as detailed elsewhere. The specific surface area, 34.9 m2 g-1, was determined by BET nitrogen adsorption using a 30/70 N2/He mixture (Monosorb, Quantachrome Corp). HAP crystals in the form of a suspension in water (41.8 g L-1) were used for the crystal growth experiments.

Solutions were prepared using triply distilled carbon dioxide-free water and filtered before use through washed 0.22 μm filters (Millipore, Bedford, Mass.). Calcium concentrations were determined either complexometrically by EDTA titration with Eriochrome Black-T as indicator, or by atomic absorption (Perkin-Elmer, model 3100, Norwich, Conn.). Carbon dioxide-free potassium hydroxide solutions were prepared from washed Reagent grade pellets in a nitrogen atmosphere.

Crystal growth experiments were made in magnetically-stirred water-jacketed Pyrex vessels at 37.0±0.05° C. with ionic strength, I=0.15 mol L⁻¹, adjusted by the addition of sodium chloride. Supersaturated solutions were prepared by introducing calcium chloride solution, followed by potassium dihydrogen phosphate solution. The pH was adjusted to the required value by the slow addition of potassium hydroxide solutions. During the reactions, carbon dioxide was excluded by bubbling with presaturated nitrogen gas. After equilibration, 0.5 mL of the HAP slurry was introduced to initiate the reaction. Since the nucleation and growth of crystals consume solution lattice ions, the lowering of pH was used to trigger the addition of two titrant solutions that served to maintain constant the pH, the concentrations of calcium and phosphate and the ionic strength of the solution. A glass electrode (Orion, model 9101), standardized using two NBS buffer solutions at pH=7.386 and 4.028 at 37° C., was used to control titrant addition through a potentiostat. The total calcium concentration in all experiments was 6.00×10⁻⁴ mol L⁻¹ with a calcium/phosphate molar ratio of 1.67, so as to achieve a supersaturation with respect to HAP of σ=5.55 (as defined in Eq. (1)), as computed from mass balance, proton dissociation, electroneutrality, and equilibrium expressions involving calcium and phosphate species.

σ=S−1=[IP/K_{SO}]^{1/v}−1 (−)

In Eq. (1), v is the number of ions per formula unit of precipitating phase and IP and K_{SO} are, respectively, the ionic and solubility products of HAP. The addition of the fullerene derivatives at micromolar levels to the reaction solutions did not affect the established supersaturation.

During the reactions, samples were withdrawn periodically, filtered (0.22 μm Millipore filter) and analyzed for calciwn by atomic absorption and for phosphate spectrophotometrically (Varian, Cary 210) as the phosphovanadomolybdate complex in order to verify the constancy of the solution composition. Solid phases were examined by X-ray powder diffraction, XRD, (Siemens Nicolet/Nic spectrometer, CuK radiation with Ni filter=1.540; 2 from 3o to 45o), by scanning electron microscopy (SEM at 20 kV; JEOL JSM-5300, Noran Instrumental Inc. Middleton, Wis.) and by diffuse reflectance infared fourier transform spectroscopy (FTR, Perkin Elmer 1760×Fr-IR spectrometer). Plots of mineralization were calculated from plots of titrant volumes as a function of time as described previously.

In order to investigate the uptake of fullerene derivatives by HAP surfaces, an equilibrium adsorption experiment was performed in which 0.0209 g of HAP in its saturated solution was equilibrated with various concentrations of these additives. A UV-visible spectrophotometer (Perkin-Elmer model 3100) was used for the analysis of fullerene derivative concentrations. Since the ζ-potential of HAP surfaces was markedly influenced by the presence of the additives, this parameter was used as an indication of the extent of adsorption. The ζ-potential of HAP surfaces for the same suspensions in the presence of these additives was measured using a Malvem Zetasizer IIc (Malvern, England).

Syntheses

1,2-dihydro-1,2-methanofiullerene[60]-61-carboxylic Acid, CM-CHCOOH

1,2-Dihydro-1,2-methanofullerene[60]-61-carboxylic acid was synthesized according to the procedure by Isaacs and Diederich.

Tetraethyl Ethenylidenebisihosphonate

In a typical synthesis, paraformaldehyde (2.60 g, 86.7 mmol) and diethylamine (1.79 mL, 1.27 g, 17.4 mmol) were combined in MeOH (25 mL) with gentle warming to aid dissolution. The mixture was then cooled to room temperature and 5.0 g (17.3 mmol) tetraethyl methylenediphosphonate was added with stirring. The reaction was refluxed for 24 hours under atmospheric conditions and then diluted with 25 mL MEOH and concentrated under vacuum at 35° C. Toluene (50 mL) was added to the flask and the contents were again concentrated under vacuum at 35° C. This step was repeated a second time to ensure that all of the MeOH had been removed. The intermediate (tetraethyl 2-methoxyethylenebisphosphonate) was then placed under vacuum at room temperature for 3 h. 1H NMR (250 MHz, CDCl3): 4.15 (m, 8H), 3.84 (td, 2H, JP-H=16.3, JH-H=5.4), 3.33 (s, 3H), 2.65 (tt, 1H, JP-H=23.8, JH-H=5.4), 1.30 (t, 12H, J=7.0).

The reaction flask was attached to a septum-capped soxlet extractor containing 4 Å molecular sieves and the entire system was flushed with Ar for 15 minutes. Approximately 125 mL anhydrous toluene was then added through the condenser. p-Toluenesulfonic acid monohydrate (0.20 g, 1.1 mmol) was added under Ar sparge and the flask was wrapped with aluminum foil. The reaction was refluxed under inert atmosphere for 14 h. After solvent removal, the remaining light yellow oil was redissolved in CHCl₃, washed with three 50 mL portions of DI H2O, and dried over MgSO4. The CHCl₃ was then removed, leaving a yellow oil that distilled over at 139° C./1 torr as a colorless liquid. An analytically pure sample was prepared by column chromatography on SiO2 with 50:50 hexanes/acetone eluent. Yield: 4.0 g, 77%. 1H NMR (250 MHz, CDCl₃): 6.96 (distorted dd, JP-H=37.6, JP-H=34.1), 4.14 (m, 8H), 1.33 (t, JH-H=7.1, 12H). 13C NMR (250 Mfz, CDCl₃, TMS ref): 149.28, 131.80 (t, JP-C=166.5), 62.60 (t, JP-C=2.8), 16.20 (t, JP-C=3.3). 31P NMR (250 MHz, CDCl₃): 13.73 ppm downfield from H3PO4. IR (neat): 2984 (C—H), 2935 (C—H), 2910 (C—H), 1636 (C═C), 1251, (P═O), 1024 (C—O), 974 (P—C—P bend), 803 cm⁻¹ (P—O).

Benzylidene Glycine Ethyl Ester

In a typical synthesis, the hydrochloride salt of glycine ethyl ester (15.0 g, 107.5 mmol) was dissolved in 125 mL CH₂Cl₂. Treatment with freshly distilled NEt3 (21.8 g, 30 mL, 215.8 mmol) resulted in the formation of a small amount of white precipitate. Benzaldehyde (7.60 g, 7.28 mL, 71.6 mmol) was then added to the reaction at room temperature followed by 6 g MgSO4 to remove the water by-product. After 10 h stirring, the solution was filtered and reduced under vacuum to give a yellow oil. This compound was dissolved in 100 mL Et₂O, washed with sat. aq. NaCl (6×50 mL) and dried (MgSO₄). Solvent removal under vacuum at 25° C. gave a light yellow oil that was used without further purification. The compound was stored at 10° C. Yield: 13.42 g, 98%. 1H NMR (250 MHz, CDCl₃, TMS ref): 8.30 (s, 1H), 7.78 (m, 2H), 7.42 (m, 3H), 4.40 (s, 2H), 4.24 (q, 2H, J=7.1), 1.31 ppm (t, 3H, J=7.1). ¹³C NMR (250 MHz, CDCl₃): 169.99, 165.27, 135.45, 131.07, 128.46, 128.35, 61.94, 60.94, 14.07 ppm. IR (neat): 3063 (Ar C—H), 3029 (Ar C—H), 2983 (C—H), 2938 (C—H), 2975 (C—H), 2903 (C—H), 2854 (C—H), 1735 (C═O), 1646 (C═N), 1200 (C—O), 1027 (C—O), 759 (Ar-H out-of-plane), 694 cm⁻¹ (Ar-H out-of-plane).

Ethyl N-Benzylidene-2-amino-4,4-bis(diethoxylihosphoryl)butyrate

In a typical synthesis, benzylidene glycine ethyl ester (1.91 g, 10.0 mmol) was added to a solution of NaOEt (1 mmol) in 20 mL of fresh absolute EtOH at −8° C. (ice-salt bath). Tetraethyl ethenylidenebisphosphonate (3.00 g, 10.0 mmol) was added drop-wise over 3 minutes with vigorous stirring. The reaction was stirred for 30 min at 25° C. and then neutralized with sat. aq. NH4Cl (ca 3 mL). Removal of the EtOH (ca. 25° C.) at reduced pressure left a paste-like residue that was extracted with CHCl₃ (3×20 mL). The organic fraction was dried over MgSO4, filtered and evaporated at reduced pressure to give a light yellow oil that was stored in the freezer. Yield: 4.72 g, 96%. 1H NMR (250 MFz, CDCl₃, TMS ref): 8.38 (s, 1H, N=CHPh), 7.76 (m, 2H), 7.44 (m, 3H), 4.46 (dd, 1H, J=4.9, J=8.8), 4.16 (m, 10H), 2.7-2.3 (m, 3H), 1.32 (m, 15H). ¹³C NMR (250 Miz, CDCl₃): 171.08, 165.47, 135.54, 131.18, 128.49, 128.44, 69.81 (dd, J=4.4, J=10.5), 62.5 (m), 61.11, 32.73 (t, J=133.1), 28.83, 16.30 (d, 3JC-P24.3), 14.05 ppm. 31P NMR (250 MHz, CDCl₃): 23.66, 23.93 ppm, downfield from H₃PO₄. IR (neat): 2984 (C—H), 2933 (C—H), 2907 (C—H), 2872 (C—H), 1737 (ester C═O), 1641 (CH═N), 1280 (shoulder, P═O), 1253 (P═O), 1027 (C—O), 970 (P—C—P), 754 cr1 (P—O).

Ethyl 2-Amino-4,4-bis(diethoxyphosphoryl)butyrate

In a typical synthesis, ethyl N-benzylidene-2-amino-4,4-bis(diethoxyphosphoryl)butyrate (3.00 g, 6.1 mmol) was dissolved in 10 mL DI H2O at room temperature. p-Toluenesulfonic acid monohydrate (1.74 g, 9.2 mmol) was then added and the mixture was stirred for 30 min. at room temperature. After extraction with Et₂O (4×15 mL) to remove benzaldehyde, the aqueous solution was rendered alkaline by addition of saturated aqueous NaHCO₃ (ca. pH 8.1) and then extracted with CHCl₃ (4×10 mL). The organic layer was dried over MgSO4, filtered and evaporated under reduced pressure to give a light yellow oil that was used without further purification. The compound was stored in the freezer and checked by NMR to ascertain purity before each use. Yield: 2.3 g, 94%. 1H NMR (200 MHz, CDCl₃, TMS ref.): 4.2 (m, 10H), 3.77 (dd, J=4.4, J=10.6, 1H), 3.14-2.84 (dddd, 3JHH=3.3, 3J′HH=8.9, 2JHP=23.2, and 2J′HP-24.7 Hz, 1H, PCHP), 2.5-2.2 (m, 1H), 2.1-1.8 (m, 1H), 1.3 ppm (m, 15H). ¹³C NMR (200 Mz, CDCl₃): 175.50, 62.5 (m), 61.02, 52.77 (dd, J=10.3, J=3.1), 32.77 (t, J=133.9), 30.49, 16.39 (d, J=6.1), 14.22 ppm. 31P NMR (250 MHz, CDCl₃,): 24.43, 24.16 ppm, downfield from H₃PO4. IR (neat): 3476 (br, N—H), 2984 (C—H), 2934 (C—H), 2910 (C—H), 1733 (ester C═O), 1646 (NH2 deformation), 1277 (shoulder, P═O), 1245 (P═O), 1020 (C—O), 972 (P—C—P), 838 (P—O), 799 cm−1 (P—O).

Ethyl 4.4-bis(diethoxyphosphoryl)-2-(1,2-dihydro-1,2-methanofullerene[60]-61-carboxamido)butyrate, C₆₀AMBP-protected

C₆₀-CHCOOH (0.100 g, 0.128 mmol) and 1-hydroxybenzotriazole, BtOH, (0.035 g, 0.256 mmol) were combined in 30 mL PhBr. Approximately 0.104 g (0.256 mmol) ethyl 2-amino-4,4-bis(diethoxyphosphoryl)butyrate, AMBP, was added via pipette. Immediately, 0.0530 g (0.256 mmol) 1,3-dicyclohexyl-carbodiimide (DCC) was added to the reaction flask. Stirring for 5 d at room temperature under inert atmosphere yielded a dark cranberry colored solution. The product was purified by column chromatography on a 10×1 in. SiO2 column using toluene to elute PhBr and a 1% MeOH/CHCl₃ solution to elute the cranberry colored product. Two precipitations from CHCl₃ with Et₂O gave a brown solid that was collected in a centrifuge tube and dried overnight under vacuum at room temperature. Yield: 0.103 g, 69%. 1H NMR (200 MHz, CDCl₃): 8.93 (br d, 1H, J=7.8), 4.85 (m, 1H), 4.79 (s, 1H), 4.27 (m, 10H), 2.71-2.46 (m, 3H), 1.43 ppm (m, 15H). ¹³C NMR (200 MHz, CDCl₃): 170.74, 165.37, 34 out of 60 flullerene resonances observed—148.53, 148.36, 146.48, 146.17, 145.59, 145.50, 145.08, 144.89, 144.80, 144.61, 144.43, 144.31, 144.24, 144.02, 143.76, 143.70, 143.49, 143.44, 143.10, 142.86, 142.77, 142.72, 142.55, 142.24, 141.98, 141.91, 141.85, 140.87, 140.58, 140.20, 140.10, 136.20, 136.10, 71.56 (2×; sp3 C₆₀ bridgehead carbon atoms), 63.33 (m), 61.93, 53.13, 41.11, 33.34 (t, J=132.1 Hz), 26.72 (br s), 16.48 (br d), 14.23 ppm. 31P NMR (400 MHz, CDCl₃, H₃PO₄): 24.51 (d, J=5.2 Hz), 24.48 ppm downfield from H₃PO₄ (d, J=5.2 Hz). IR (KBr): 3244 (N—H), 2977 (C—H), 2900 (C—H), 1734 (carboxy ester C═O), 1684 (amide I band), 1540 (amide II band), 1249 (P═O), 1023 (C—O), 972 (P—C—P), 798 (P—O) 528 cm⁻¹ (fullerene stretch). UV-vis (CHCl₃, λ run, (ε M⁻¹ cm−1)): 326 (3.4×104), 402 (4.1×103), 416 (3.4×103), 427 (3.7×103), 479 (1.7×103), 690 (2.1×102). High res. APCI-MS (50:50 CHCl₃/MeOH): 1164.153200 (M++1). Anal Calc. for C76H₃1NO9P2: C, 78.42; H, 2.68; N, 1.20; O, 12.37; P, 5.32. Found: C, 77.42; H, 3.19; N, 1.19; (O, 12.89); P, 5.31.

4,4-bisphosohono-2-(1,2-dihydro-1,2-methanofullerene[60]-61-carboxamido)butyric acid, C₆₀AMBP

C₆₀-AMBP-protected as described above (0.160 g, 0.137 mmol) was dissolved in 125 mL anhydrous toluene in a glove box. I-Si(CH₃)₃ (0.196 mL, 0.275 g, 1.37 mmol) was added drop-wise at room temperature over 30 seconds. After 72 hours stirring at 50° C., the reaction was filtered to remove a dark insoluble product. The filtrate was then removed from the glove box and quenched with 1 mL MeOH. An insoluble brown precipitate formed immediately. It was collected in a centrifuge tube, washed consecutively with CHCl₃ and Et₂O, and then dried under vacuum at 65° C. for 8 hours. This compound was insoluble in all common solvents. Yield: 127 mg, 90%. IR (KBr): 3600-2500 (br, P—OH), 2923 (C—H), 1719 (Acid C═O), 1650 (amide I band), 1540 (amide II band), 1241-1024 (envelope P═O, C—O, OH bend), 928 (P—C—P), 526 cm−1 (fullerene resonance).

The intermediate tetra-silyl ester, produced by the reaction of ITMS with the four phosphonate ester groups was characterized by 1H NMR. 1H NMR (200 MHz, CD2Cl2): NH not observed, 5.26 (s, 1H), 5.04 (m, 1H), 4.37 (q, 2H), 2.75-2.15 (br m, 3H), 1.35 (t, 3H), 0.52 and 0.43 (br s, 36H). CH₃CH₂I is also present in the sarnple as a by-product of the deprotection reaction.

4,4-bisphosphono-2-(polyhdroxyl-1,2-dihdro-1,2-methanofullerene[60]-61-carboxamido)butyric Acid, C₆₀(OH)₁₆AMBP

C₆₀AMBP as described above (0.050 g, 0.05 mmol) was dissolved in 0.5 mL 40% tetra n-butylammonium hydroxide and then diluted to 10 mL with 1 M KOH. The mixture as stirred for 24 h at room temperature and then chromatographed on Sephadex G-25 fine) size exclusion gel (2.5″×5″). The product eluted as a well-defined brown-orange and followed later by a volume of colorless basic eluent. Between aliquots, the column as rinsed thoroughly with DI H₂O until the pH of the eluent was no longer basic. After three passes, the pH of the collected sample fractions was ca. 6, suggesting that most of the base had been removed. The collected fractions were reduced to 10 mL under vacuum with very gentle heating (T<30° C.) and then dried under slow air flow overnight to remove the remaining solvent. The resulting flaky, black solid was collected and dried at 100° C./1 torr over P2O5 for 12 hours. 37 mg C₆₀(OH)₁₆AMBP isolated. ¹H NMR (250 MHz, D₂O): 4.15, 3.95, 3.56, 2.25, 1.9-0.9 ppm (all br signals). 31P NMR (250 MHz, D20): 12.4 ppm upfield from H₃PO₄ (br weak singlet). IR (KBr): 3358 (v br, O—H), 2922 (aliphatic C—H), 1717 (shoulder, carboxy C═O), 1595 (v br, amide C═O), 1387 (v br, O—H bend), 1239 (P═O), 1072 (s, C—O), 1045 (s, C—O). UV-vis (H₂O): No maxima were observed; the absorption curve decreases gradually toward the visible region. To describe the absorption strength, measurements were taken at 300, 400, and 500 nm. The molar extinction coefficients at these wavelengths are 22.9×106, 7.57×106 and 2.08×106 cm²/mol, respectively. MALDI-MS: no peaks observed other than 720 (C₆₀). Anal. Calc. for C₆₆H₂₇NO₂₅P₂ (C₆₀(OH)₁₆AMBP) C, 61.18; H, 2.10; N, 1.09; P, 4.78; O, 30.87 found: C, 60.38; H, 2.77; N, 1.27; P, 4.86; (0, 30.72 by difference). Analysis for potassium showed only a trace amount (<0.37%). This product, C₆₀(OH)₁₆AMBP, is one of two bone-targeted fuillerenes tested in this Example.

Fullerenol[60], C₆₀(OH)₃₀.2H₂O

In a typical fullerenol synthesis, 20 mg (2.7×10-5 mol) C₆₀ (99.5%) was dissolved in a minimal volume of toluene (ca. 20 mL), using sonication to encourage dissolution. This solution was then stirred vigorously with 10 mL concentrated KOH (ca. 1 g/mL) and three drops tetra-n-butylammonium-hydroxide (TBAH) phase transfer catalyst. Decoloration of the toluene layer occurred within minutes accompanied by formation of a black semi-solid at the solvent interface. The toluene layer was carefully decanted and the remaining water layer was sonicated briefly and then stirred under air sparge at room temperature for 10-12 hours to allow further reaction and to remove remaining toluene. Deionized water was then added to the remaining solution or solid to bring the total volume to 25 mL. The resulting suspension was stirred for 2448 hours to ensure complete reaction. The resulting orange-brown solution was diluted with an additional 10 mL DI H2O and then vacuum filtered through celite on a Buichner funnel to remove a small amount of insoluble material. The filtrate was concentrated under reduced pressure with gentle warming until precipitate started to form. MEOH was then added to complete precipitation. A mixture of brown and white solids was collected by centrifugation. The solids were redissolved in a minimal amount of water and again precipitated with MEOH to remove additional KOH and TBAH impurities. This step was repeated a third time. After the final precipitation, the compound was chromatographed on Sephadex G-25 (fine) size-exclusion gel. Two bands were observed: the first broad, orange band was collected while the second brown-orange band, being strongly basic, was discarded. The collected eluent was concentrated and precipitated with MeOH. If a pH measurement of the mother liquor indicated presence of base (if the pH>6 when saturated with CO2), the compound was further purified. UV-vis: No maximna were observed; the absorption curve decreases gradually toward the visible region. To describe the absorption strength, measurements were taken at 300, 400, and 500 nm. The molar extinction coefficients at these wavelengths are 29.3×106, 9.7×106 and 2.6×106 cm2/mol, respectively. Anal Calc. for C₆₀H₃₀O₃₀ [symbol] 2H₂O, (C₆₀(OH)₃₀ [symbol] 2H2O) C, 56.88; H, 2.71; O, 40.41. Found: C, 56.54; H, 2.53; (O, 40.93 by difference). This product, C₆₀(OH)₃₀, is the second of two bone-targeted fullerenes tested in this Example.

Results And Discussion

Constant composition growth experiments were first performed in pure supersaturated solutions of HAP and then in the presence of C₆₀(OH)₁₆AMBP and C₆₀(OH)₃₀. The results are presented in Table 1.

TABLE 1

| C₆₀(OH)₃₀/ | Rate/ | C₆₀(OH)₁₆AMBP/ | Rate/10⁻⁸ |
|---|---|---|---|
| 10⁻⁶ mol L⁻¹ | 10⁻⁸ mol min⁻¹ m⁻² | 10⁻⁶ mol L⁻¹ | mol min⁻¹ m⁻² |
| 0 | 9.09 | 0 | 9.09 |
| 0.41 | 8.31 | 0.39 | 7.67 |
| 0.81 | 7.8 | 0.77 | 6.51 |
| 1.22 | 6.55 | 1.16 | 4.55 |
| 1.63 | 5.94 | 1.54 | 3.77 |
| 2.03 | 5.7 | 1.93 | 2.83 |
| 2.44 | 4.65 | 2.32 | 2.46 |
| 2.85 | 4.07 | 2.7 | 1.92 |
| 3.25 | 3.97 | 3.09 | 1.57 |
| 3.66 | 3.85 | 3.47 | 1.23 |
| 4.07 | 2.64 | | |
| 4.47 | 2.49 | | |

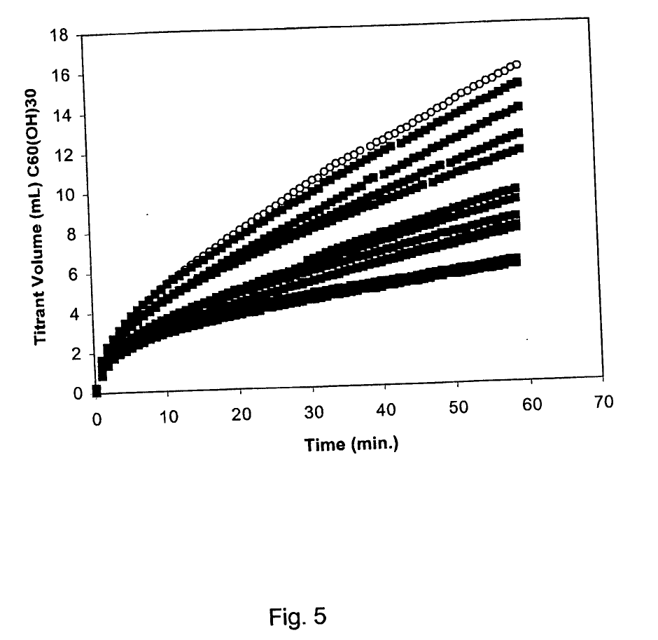

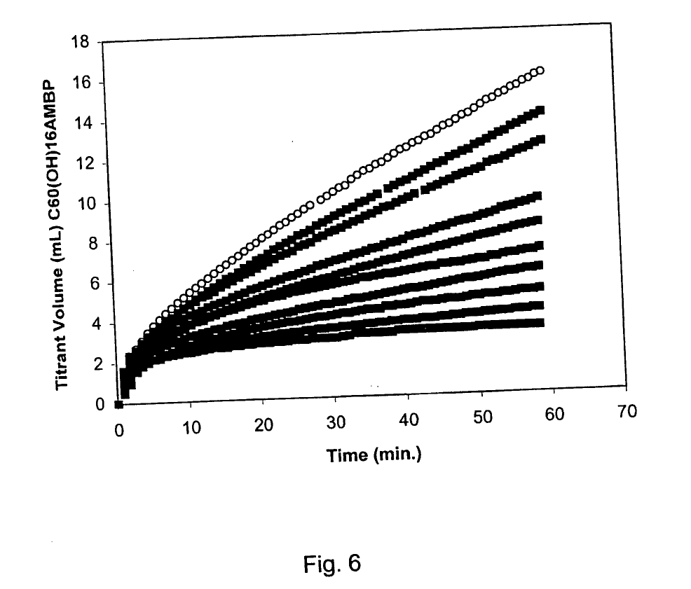

FIGS. 5 and 6 show typical plots of titrant volume required to maintain the supersaturation as a function of time at different fullerene concentrations for C₆₀(OH)₃₀ and C₆₀(OH)₁₆AMBP, respectively. For comparison, a curve of titrant consumption for the growth of HAP crystals in pure supersaturated solutions is included in each plot (open symbols). All the rate curves show a rapid titrant addition immediately following the introduction of seed crystals. This frequently observed phenomenon, usually attributed to conditioning of the surface of the seed crystals in the supersaturated solution, may reflect ion exchange involving solution and surface cations and protons, or the removal of active growth sites on the seed crystals due to the rapid crystallization of high energy sites. Significant reductions of the initial surges observed in the presence of the fullerenes suggest their adsorption at the growth sites on the HAP seed crystals, thus their competition with other initial surface processes. It can be seen in FIG. 5 that linearity of the rate plots of HAP crystal growth (reflected by the constant slopes of volume versus time curves) was usually achieved 10-20 minutes after seed introduction to the supersaturated solution in experiments performed in the absence and presence of fullerenes. It should be noted that at high concentrations of inhibitors, HAP crystals appear to grow only during the initial reaction stage.

It is quite well established that strong inhibitors of crystal growth, such as the phosphonates, act by blocking, through adsorption, active growth sites at the crystal surfaces. Commonly, inhibition kinetics data are interpreted in terms of a simple Langmuir adsorption model. Assuming that the adsorbed fullerene molecules occupy a fraction, θ, of the active growth sites, thereby preventing them from participating in the growth, the growth rate R can be written in terms of the uninhibited rate Rₒ (Eq. (2)):

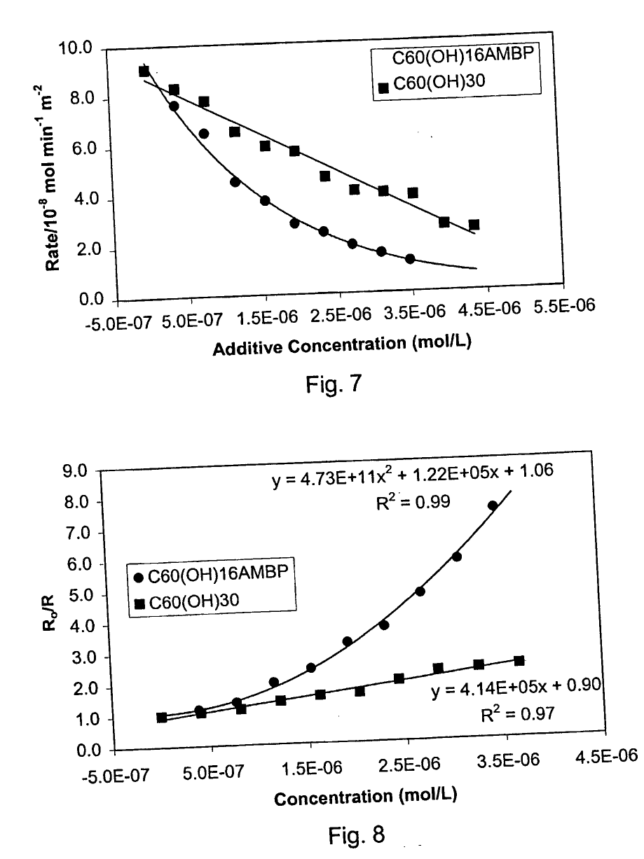

Rₒ/R=1+KLC (2)

in which KL is the adsorption affinity constant with units of liters per mole. HAP crystal growth rates R measured in the presence of C₆₀(OH)₃₀ and C₆₀(OH)₁₆AMBP are plotted in FIG. 7 as a function of additive concentration.

Comparison of the two curves shows that both compounds inhibit HAP crystal growth, with C₆₀(OH)₁₆AMBP being the more effective inhibitor. At a concentration of 3.1×10-5 M, C₆₀(OH)₃₀ reduces the HAP crystal growth by 58%, while C₆₀(OH)₁₆AMBP reduces the rate by 87%. The increase in inhibition is linear with concentration for C₆₀(OH)₃₀, but not for the bisphosphonate derivative, C₆₀(OH)₁₆AMBP.

Following Eq. (2), plots of Ro/R as a function of concentration for each compound are shown below in FIG. 8. The linear relation shown for C₆₀(OH)₃₀ indicates that the compound inhibits HAP crystal growth by the mechanism described by the Langmuir formalism. From the slope, the affinity constant is 4.14×105 L mol-1 (R2=0.97). In fact, this affinity of fullerenol materials for HAP has also been observed previously in an in vivo mouse model study using radiolabeled ¹⁶⁶Ho@C₆₀(OH)ₓ.

The plot in FIG. 8 of RₒR versus concentration for C₆₀(OH)₁₆AMBP lacks linearity, which suggests that the mechanism of inhibition is different from that described by the Langmuir model. The model relies on the assumptions that the inhibitor reversibly binds to the mineral surface at active growth sites and that the surface-bound molecules do not interact with one another. For C₆₀(OH)₁₆AMBP, these assumptions may be invalid. The curvature observed in FIG. 7 for C₆₀(OH)₁₆AMBP at higher concentrations may be caused by lateral interference among the surface-bound molecules. Once adsorbed to a surface, molecules are capable of lateral movement and surface aggregation. Under such conditions, an equilibrium distribution may be impossible, thereby preventing the molecules from effectively inhibiting mineralization. In experiments with higher inhibitor concentrations, molecular aggregation can develop more easily, thus making the decrease in inhibitory activity more apparent. Such aggregation may result from enhanced intermolecular hydrogen bonding interactions among the hydroxyl and carboxylic and phosphonic acid groups of the fullerene material.

Table 2 compares the percent reductions in growth rate of the two fullerenol derivatives to that for 1-hydroxyethylidene-1,1-diphosphonic acid, CH₃C(OH)[P(O)(OH)₂]₂, a commercially-available bone-vectored compound used in the treatment of osteoporosis. At 1.0×10⁻⁶ M inhibitor concentration, neither C₆₀(OH)₃₀ nor C₆₀(OH)₁₆AMBP is as effective as CH₃C(OH)[P(O)(OH)₂]₂ at inhibiting HAP crystal growth. C₆₀(OH)₃₀ also has a lower affinity constant, suggesting that the compound binds to the surface less strongly than CH₃C(OH)[P(O)(OH)₂]₂. The lower binding constant for C₆₀(OH)₃₀ is not surprising given that the fullerenol does not contain a bisphosphonic acid moiety. The hydroxyl functionalities present in C₆₀(OH)₃₀ are capable of hydrogen bonding to the surface, but they do not provide the stronger ionic interactions that are present with bisphosphonic acid groups.

TABLE 2

| | | Percent | |
|---|---|---|---|
| | | Reduction | |
| | Concentration | in Growth | Affinity Constant |
| Compound | mol/L | Rate | L mol⁻¹ |
| C₆₀(OH)₃₀ | 1.0 × 10⁻⁶ | 28 | 4.14 × 10⁵ |
| C₆₀(OH)₁₆AMBP | 1.0 × 10⁻⁶ | 50 | — |
| C₆₀(OH)₁₆AMBP | 3.5 × 10⁻⁶ | 87 | — |
| CH₃C(OH)[P(O)(OH)₂]₂ | 1.0 × 10⁻⁶ | 69^{ref 29} | 13.3 × 10^{5 ref 29} |

13.3 Comparison of C₆₀(OH)₁₆AMBP to CH₃C(OH)[P(O)(OH)₂]₂ is somewhat problematic because the latter molecule inhibits HAP crystal growth by a mechanism that appears strictly Langmuirian, with the affinity constant being independent of concentration. As shown in FIG. 7, such independence is not the case for C₆₀(OH)₁₆AMBP.

Despite uncertainty in the inhibition mechanism, the results demonstrate that both C₆₀(OH)₃₀ and C₆₀(OH)₁₆AMBP inhibit HAP crystal growth from supersaturated calcium phosphate solutions and that both compounds have relatively strong affinities for HAP. The greater crystal growth inhibition by C₆₀(OH)₁₆AMBP stresses the importance of incorporating bisphosphonate moieties into bone-vectored fullerene derivatives. Further explanation for the diminished rate inhibition observed at higher C₆₀(OH)₁₆AMBP concentrations will require studies of other fullerene compounds, especially those having multi-directional surface-binding functionalities such as C₆₀C₆(COOH)₁₂ and others. As the first investigation, however, the present study demonstrates the potential usefulness of fullerene-based materials in tissue-targeting technologies and lays the ground-work for in vivo studies using radiolabeled bisphosphonate materials as potential bone therapeutic agents that effectively target bone and are likely to be effective for reducing the rate of bone loss.

Fluorination

When one or more fluorine ions are bound to the bone-vectored fullerene disclosed above, the result is a bimodal agent having both bone-targeting and bone growth ligands. It is preferred that a suitable molecular template be used during production of the bimodal agent, so that the ligands are bound in opposing positions on the fullerene. If the ligands are too close together, the effectiveness of one or both may be reduced.

Fluorine atoms can be affixed to the fullerene by any suitable method, including exposing the fallerene soot to a fluorine gas at a pressure of several hundred Torr, and including the methods and techniques disclosed in U.S. Pat. Nos. 5,558,903 and 5,958,523, both of which are incorporated herein by reference. It is believed that the number of fluorine atoms that can be affixed to the present bisphosphonated fullerenes so as to achieve optimal bone growth stimulation is between 1 and 20.

The present invention provides advantages over previously known bisphosphonated bone therapeutic agents, in that it allows both an anti-resorption agent and a growth agent to be placed in proximity to active bone sites and does so without interfering with other biological mechanisms and without risk of toxicity.

While the present invention has been disclosed and described in terms of a preferred embodiment, the invention is not limited to the preferred embodiment. For example, while the present invention has been described for use in bone, it should be understood that the target tissue may be any suitable material containing hydroxyapatite, such as teeth. In addition, various modifications to the ligands and the scaffolding, or carrier, and the arrangement of the ligands on the scaffolding can be made without departing from the scope of the invention. In the claims that follow, any recitation of steps is not intended as a requirement that the steps be performed sequentially, or that one step be completed before another step is begun, unless explicitly so stated.

What is claimed is:

1. A therapeutic composition targeted to diseased or injured bone comprising:
a biologically inert carrier;
a bone vector chemically bonded to the carrier; and
a therapeutic agent chemically bonded to the carrier;

2. The composition of claim 1 wherein the bone vector comprises a bisphosphonate.

3. The composition of claim 1 wherein the carrier comprises a fullerene.

4. The composition of claim 1 wherein the carrier is C₆₀.

5. The composition of claim 1 wherein the carrier is C₆₀ and the bone vector comprises a bisphosphonate.

6. The composition of claim 1 wherein the carrier is C₆₀(OH)₁₆.

7. The composition of claim 1 wherein the therapeutic agent comprises fluoride.

8. A method for providing bone therapy in a patient in need of bone therapy comprising administering to said patient a pharmaceutically effective amount of a compound comprising a biologically inert carrier, a bone vector, and a therapeutic agent.

9. The method of claim 8 wherein the bone vector comprises a bisphosphonate.

10. The method of claim 8 wherein the carrier comprises a fullerene.

11. The method of claim 8 wherein the carrier is C₆₀.

12. The method of claim 8 wherein the therapeutic agent comprises fluoride.

13. The composition of claim 8 wherein the carrier is C₆₀ and the bone vector comprises a bisphosphonate.

14. A method of making a bone therapeutic agent, comprising:
providing a biologically inert carrier;
bonding a bone targeting agent to the carrier;
bonding a bone growth agent to the carrier.

15 The method of claim 14 wherein the bone targeting agent comprises a bisphosphonate.

16. The method of claim 14 wherein the carrier comprises a fullerene.

17. The method of claim 14 wherein the carrier is C₆₀.

18. The method of claim 14 wherein the therapeutic agent comprises fluoride.

19. The composition of claim 14 wherein the carrier is C₆₀ and the targeting agent comprises a bisphosphonate.

20. A method for providing bone therapy in a patient in need of bone therapy comprising administering to said patient a pharmaceutically effective amount of a compound comprising 4,4-bisphosphono-2-(polyhydroxyl-1,2-dihydro-1,2-methanofullerene[60]-61-carboxamido)butyric acid.
